(19) United States
(12) Patent Application Publication
(10) Pub. No.: US 2021/0338208 A1
(43) Pub. Date: Nov. 4, 2021

(54) 3D ULTRASOUND IMAGING WITH BROADLY FOCUSED TRANSMIT BEAMS AT A HIGH FRAME RATE OF DISPLAY

(71) Applicant: KONINKLIJKE PHILIPS N.V., EINDHOVEN (NL)

(72) Inventors: MAN NGUYEN, MELROSE, MA (US); JEAN-LUC FRANCOIS-MARIE ROBERT, CAMBRIDGE, MA (US)

(21) Appl. No.: 17/273,331

(22) PCT Filed: Sep. 4, 2019
(86) PCT No.: PCT/EP2019/073511

§ 371 Date: Mar. 4, 2021

Related U.S. Application Data
(60) Provisional application No. 62/728,291, filed on Sep. 7, 2018.

(51) Int. Cl. A61B 8/08; A61B 8/00; G01S 15/89

(52) U.S. Cl. CPC A61B 8/5253; A61B 8/5207; A61B 8/483; G01S 15/8925; A61B 8/4494; A61B 8/5223; A61B 8/466

(57) ABSTRACT

An ultrasound system produces 3D images at a high framerate of display. A volumetric region is scanned with plane wave or diverging transmit beams to insonify a large part of or even the entire volumetric region with each transmit event. To avoid the acquisition of clutter signals in the azimuth and elevation dimensions, the plane waves or diverging beams are transmitted at angles intermediate the elevation and azimuth directions. By transmitting plane waves or diverging beams at multiple different angles which are each a combination of both the elevation and azimuth directions, sidelobe clutter is reduced in the resulting compounded images.

### RELATED APPLICATION

This application claims the benefit of and priority to U.S. Provisional No. 62/728,291, filed Sep. 7, 2018, which is incorporated by reference in its entirety.

### TECHNICAL FIELD

This invention relates to ultrasound imaging systems and, in particular, to three-dimensional (3D) ultrasound imaging with broadly focused or unfocused transmit beams at a high frame rate of display.

### BACKGROUND

Two-dimensional (2D) ultrasound imaging is conventionally done by scanning a planar image field with a one-dimensional (1D) array transducer. Beams are transmitted over the image field and echoes are acquired in response to each transmission. The received echoes are beamformed by a delay-and-sum beamformer to form scanlines of coherent echo signals across the image field. A typical number of scanlines for an image may be 128-196 scanlines. The scanlines are processed by B mode or Doppler processing to form a planar image of the tissue and/or flow in the planar image field.

A similar method can be used to scan a volumetric image field for the production of a three-dimensional (3D) image of the volumetric region. Beams are again transmitted and echoes received, but this time over a full volume and not just a plane. Accordingly, it takes much longer to scan a volume for 3D imaging. If, for instance, the volume has the same elevational and azmuthal dimensions as the azimuth dimension of the planar image described above, an equivalent quality image requires 128×128 scanlines, a total of over 16,000 scanlines. Since the echo acquisition time is governed by the fixed speed of sound in the subject, the time required to acquire a full volumetric image is long and hence the framerate of display will be slow.

A solution to the slow framerate problem is to transmit beams which each insonify and return echoes from a larger region of the volume, thereby requiring fewer transmit beams to scan the entire volume and produce a 3D image. The ultimate extension of this concept is to transmit beams which insonify most or even all of the volumetric region. The tradeoff, however, is poor image resolution, as there is little, if any, transmit beam focusing. A measure which can be taken to overcome this problem is to scan the volumetric region multiple times and then combine the results, the combined scans causing an improvement in the resolution throughout the image.

But this measure still can result in a 3D image with significant image clutter, as sidelobe levels of the largely unfocused transmit beam patterns will generally be very high. The high sidelobe levels capture off-axis energy which will appear as image clutter in the final image.

### SUMMARY

The present invention advantageously enables a volumetric region with only a few broad beams which provide an improvement in the framerate of display, but without the development of excessive clutter in the resulting 3D image.

In accordance with the principles of the present invention, an ultrasound imaging system is described which produces 3D images at a high framerate of display. A volumetric region is scanned with plane wave or diverging transmit beams to insonify a large part of or even the entire volumetric region with each transmit event. To avoid the acquisition of clutter signals in the azimuth and elevation dimensions, the plane waves or diverging beams are transmitted at angles intermediate the elevation and azimuth directions. By transmitting plane waves or diverging beams at multiple different angles which are each a combination of both the elevation and azimuth dimensions, sidelobe clutter is reduced in the resulting compounded images.

In accordance with another aspect, the present invention provides a method for generating three dimensional images. In one embodiment, the method comprises transmitting plane waves or divergent waves to the target volume and to acquire ultrasonic echo signals returned from the target volume. A plurality of such waves are transmitted at different angles to the target volume. The echo signals are received from the transmissions, and the echo signals are then processed on a spatial basis. The image data produced in response to each transmission may be compounded on a spatial basis. A volume image from the compounded image data is generated. The volume image is displayed.

In the drawings:

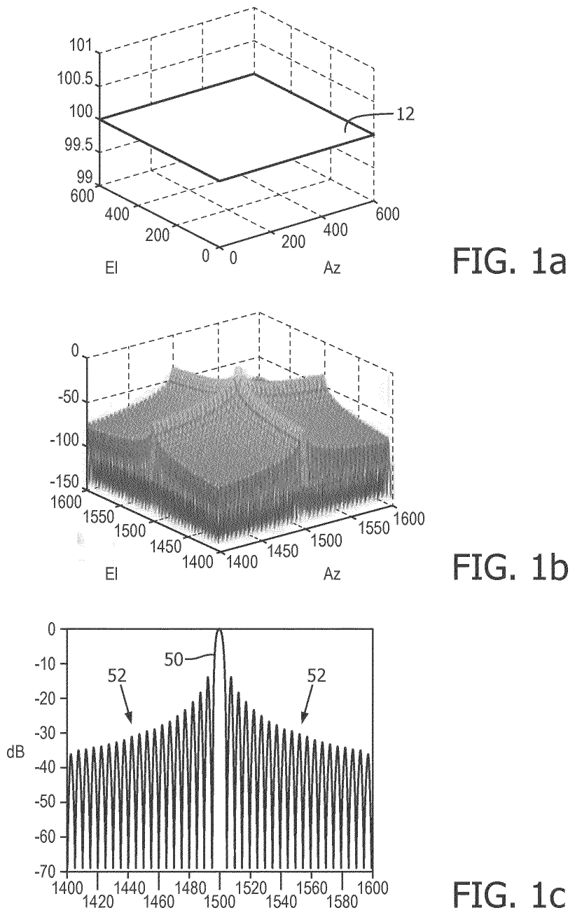

FIGS. 1a, 1b and 1c illustrate the sidelobe pattern for a two-dimensional transducer array aperture.

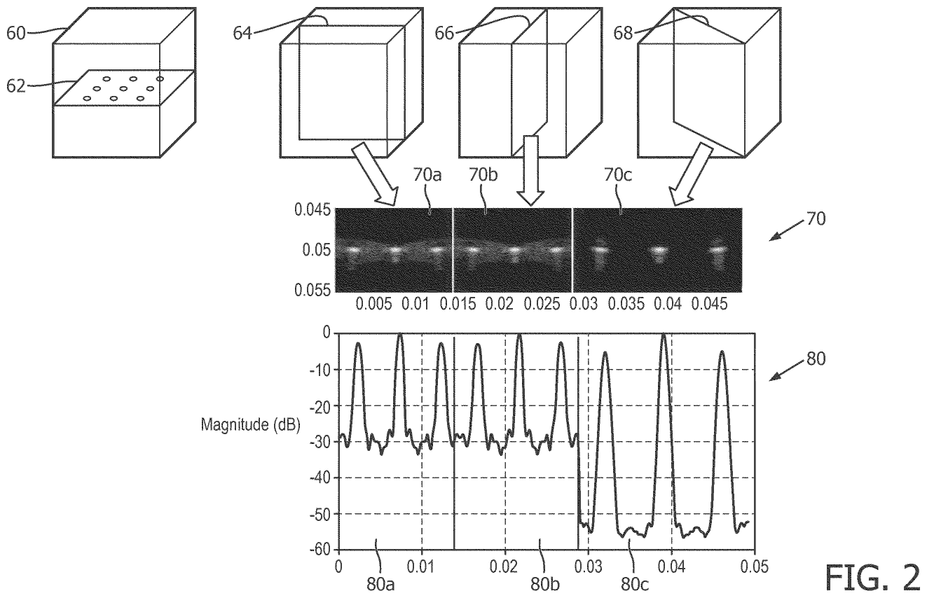

FIG. 2 illustrates the sidelobe improvement obtained by scanning a volumetric region with divergent beams at an angle intermediate the azimuth and elevation directions.

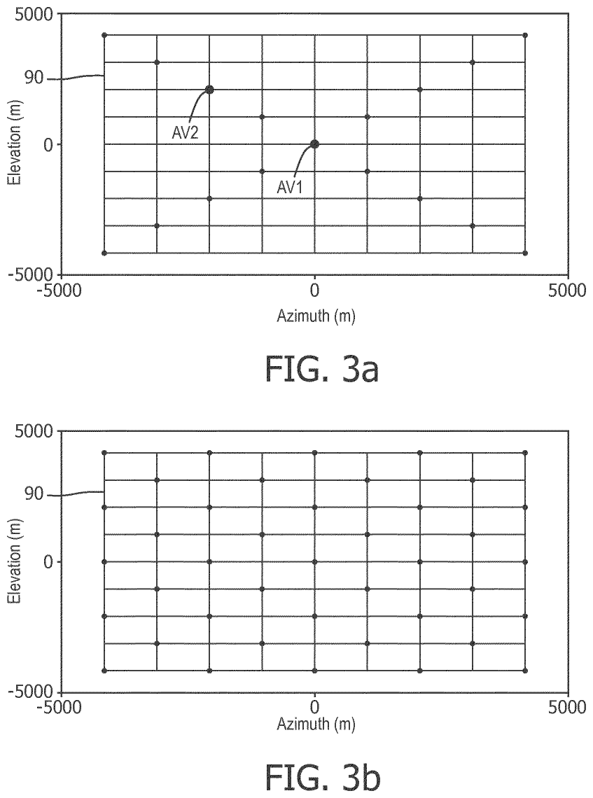

FIGS. 3a and 3b illustrate two different divergent beam scan patterns, both at angles containing both azimuth and elevation directions.

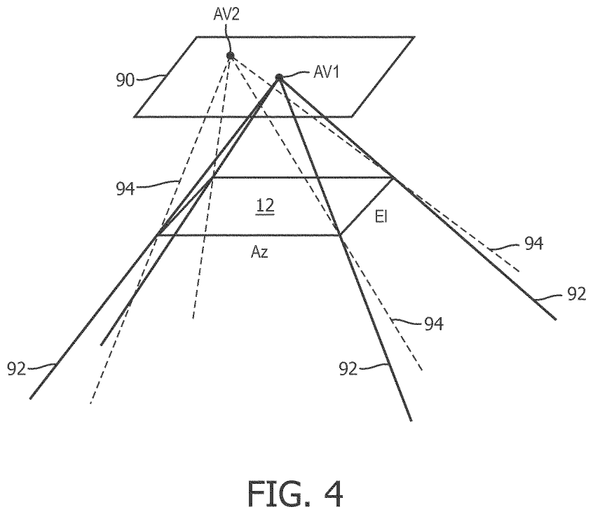

FIG. 4 illustrates divergent scan volumes for two of the apex points of FIG. 3a.

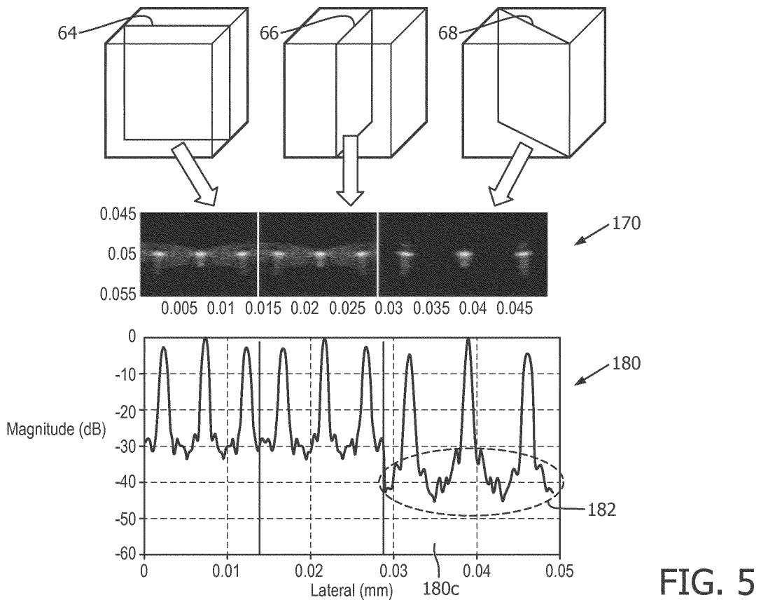

FIG. 5 illustrates the sidelobe improvement resulting from use of the divergent beam scan pattern of FIG. 3a.

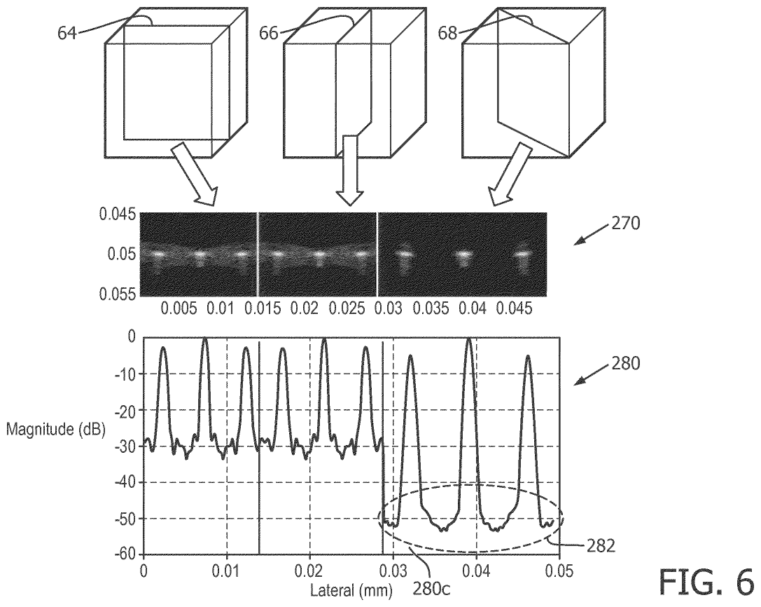

FIG. 6 illustrates the sidelobe improvement resulting from use of the divergent beam scan pattern of FIG. 3b.

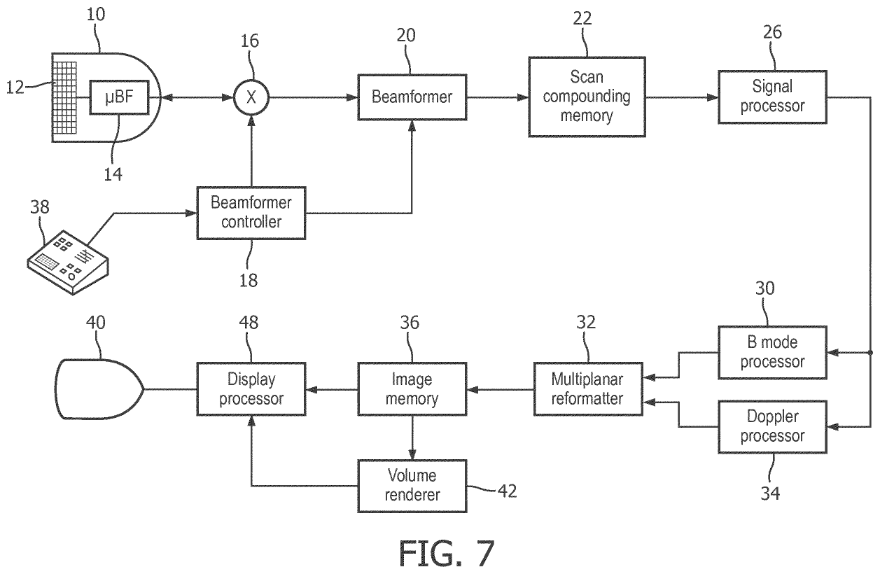

FIG. 7 illustrates in block diagram form an ultrasound imaging system constructed in accordance with the principles of the present invention.

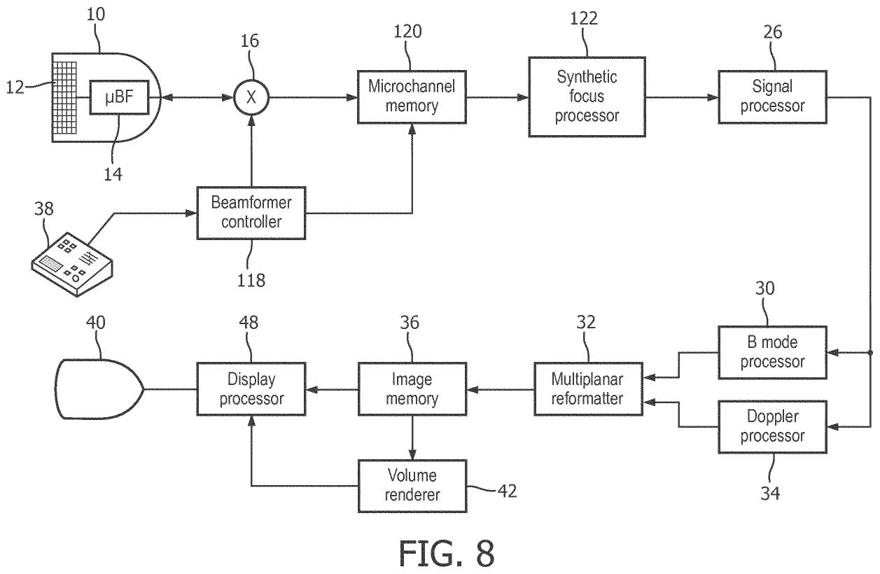

FIG. 8 illustrates in block diagram form a second ultrasound imaging system constructed in accordance with the principles of the present invention.

FIG. 1a is a perspective view of the aperture of a two-dimensional array 12 of transducer elements having rows and columns of elements extending in the azimuth (Az) and elevation (El) dimensions. The beam pattern of such an array is the Fourier complement of its aperture, shown graphically in perspective in FIG. 1b. As the beam pattern illustrates, the dominant lobes of the beams are aligned in the elevation direction of columns of elements, and in the azimuth direction of rows of elements. A cross-section taken through one of these dominant directions is shown in FIG. 1c. This plot illustrates the central main lobe 50, flanked on either side by descending patterns of sidelobes 52. The energy of the desired main lobe is seen to be accompanied by an appreciable amount of off-axis energy captured by the many sidelobes 52 of significant amplitude. It is desirable to reduce the levels of these sidelobes to reduce clutter in the ultrasound images.

Sidelobe levels can be reduced when the transmit angles of plane-wave or divergent beam transmission are neither azimuthal or elevational, but intermediate the two, such as diagonal to the two reference dimensions. The resulting beam pattern would thereby be diagonal across the transmit beam pattern of FIG. 1b. FIG. 2 demonstrates this effect with reference to an ultrasound phantom 60, containing nine point-target reflectors in a central horizontal plane 62. When this phantom is scanned by a nine by nine sequence of diverging beams, 81 transmissions in all from 81 separate and evenly spaced transmit volume apex points, an image is formed of a central azimuth plane 64 of the phantom as illustrated by ultrasound image 70a on the left side of image panel 70. The bright spots in the image are the center row of three reflectors in the phantom, and are seen to have an appreciable amount of clutter between the targets due to the high sidelobe levels. A beamplot of this azimuth plane is shown in the left illustration 80a of beamplot panel 80, which shows the three peaks of the target reflectors with intermediate sidelobe levels around −30 dB. Ultrasound image 70b and beamplot 80b show similar results for an image of the three target reflectors in the central elevation plane 66 of the phantom.

But when an image is formed of a diagonal plane 68 of the phantom, which is aligned diagonally across the array aperture, the resultant sidelobes are significantly lower, with levels below −50 dB in the right beamplot 80c. As a result, the three point-targets in the diagonal plane 68 have much lower clutter levels as shown by rightmost ultrasound image 70c in image panel 70.

A grid 90 of the transmit beam locations used to produce the experimental results of FIG. 2 is shown in FIGS. 3a and 3b. The results of FIG. 2 were obtained by transmitting a sequence of eighty-one diverging beams from a 2D array aperture 12, with an apex of each diverging beam being a virtual apex located behind the surface of the array so that the resulting diverging beam is of the form of a truncated pyramid. The eighty-one diverging beams had their apexes located at each horizontal and vertical line intersection of the grid 90. The shapes of two of the beam volumes demarcated by the large dots AV1 and AV2 are shown in FIG. 4. The apex AV1 of one diverging beam volume is located on the grid 90 behind the 2D array aperture 12 as shown in the drawing. This point is centrally located relative to the aperture as point AV1 in FIG. 3a shows, which causes the truncated pyramid of diverging beam energy to be symmetrically positioned relative to the aperture as shown in FIG. 4. Solid lines 92 mark the edges of the pyramidal beam volume. If a center line were drawn downward from the pyramid apex AV1, it would extend from the center of the 2D array 12 and normal to the surface of the array. The pyramid of diverging beam energy of the AV2 diverging beam, being on a diagonal toward the back left corner of the grid as shown in FIG. 4, results in a beam angled relative to the AV1 beam, as is seen by the dashed lines 94 marking the edges of the AV2 pyramidal beam volume. The entire AV2 diverging beam is thus steered in a different direction and angles relative to the AV1 diverging beam. While the center line of the AV2 pyramid is directed toward the center of the volumetric image field, it nonetheless extends from a different point of the array surface than that of the AV1, and at a different (non-orthogonal) angle. It is these angular differences of the diverging transmit beams which result in lower sidelobe levels of the resultant image when the echoes received from the diverging beam transmissions are compounded.

In FIG. 3a, seventeen of the grid intersection points in diagonal directions across the grid 90 demarcate the virtual apexes of seventeen diverging plane waves transmitted from a corresponding 2D array aperture 12. As FIG. 4 illustrates, the seventeen plane waves will be transmitted at seventeen different angles relative to the surface of the aperture. When seventeen such diverging plane wave beams are transmitted and their resulting echoes acquired by the array and coherently combined on a volumetric spatial basis, images of the corresponding azimuth 64, elevation 66, and diagonal 68 planes of the phantom 60 are produced as shown by image panel 170 in FIG. 5. The corresponding beam plots of the three images are shown in panel 182, where the beamplot 180c for the diagonal plane shows sidelobe levels down around −40 dB, which are circled by 182 in the drawing.

The grid 90 of FIG. 3b shows an intermediate sequence of forty-one transmit events evenly distributed across the grid and in a diagonal relationship to each other, resulting in plane wave diverging beams with forty-one different transmit angles. When the phantom 60 is scanned with this scan sequence and the same three reference planes 64, 66, and 68 are imaged, the images appear as shown in image panel 270 of FIG. 6. With forty-one different transmit volume angles, the sidelobe levels of the diagonal plane are down around −50 dB as circled at 282 in panel 280c, approaching the results for the eighty-one transmit event sequence shown in FIG. 2.

Referring now to FIG. 7, an ultrasonic diagnostic imaging system constructed in accordance with the principles of the present invention is shown in block diagram form. A two-dimensional array of transducer elements 12 is provided in an ultrasound probe 10 for transmitting ultrasonic waves and receiving echo information. The transducer array 12 is capable of scanning in three dimensions, with beams steering in both elevation and azimuth. The transducer array 12 is coupled to a microbeamformer 14 in the probe which controls transmission and reception of signals by the array elements. Microbeamformers are probe integrated circuits capable of transmit beam steering and at least partial beamforming of the signals received by groups or “patches” of transducer elements as described in U.S. Pat. No. 5,997,479 (Savord et al.), U.S. Pat. No. 6,013,032 (Savord), U.S. Pat. No. 6,623,432 (Powers et al.) and U.S. Pat. No. 8,177,718 (Savord). The microbeamformer is coupled by the probe cable to a transmit/receive (T/R) switch 16 which switches between transmission and reception and protects the main beamformer 20 from high energy transmit signals. The transmission of plane waves or diverging ultrasonic beams from the transducer array 12 under control of the microbeamformer 14 is directed by a beamformer controller 18 coupled to the T/R switch and the main beamformer 20, which receives input from the user's operation of the user interface or control panel 38. Among the transmit characteristics controlled by the transmit controller are the focus, number, spacing, shape, amplitude, phase, frequency, polarity, and diversity of transmit waveforms. Beams formed in the direction of pulse transmission may be steered straight ahead from the transducer array, or at different angles on either side of an unsteered beam for a wider sector field of view. For the 3D imaging techniques described above, unfocused plane waves or diverging beams are used for transmission.

The echoes received by a contiguous group of transducer elements (a “patch”) are beamformed by appropriately delaying them and then combining them in the microbeamformer 14. The partially beamformed signals produced by the microbeamformer 14 from each patch are coupled to a receiver in the form of a main beamformer 20 where partially beamformed signals from individual patches of transducer elements are combined into received scanlines of fully beamformed coherent echo signals from throughout a scanned target volume. Preferably the beamformer 20 is a multiline beamformer which produces multiple receive scanlines from the echoes received after a transmit event. For example, the main beamformer 20 may produce hundreds or even thousands of appropriately steered and spaced received scanlines from an insonified target volume.

The coherent echo signals of the scanlines received from each plane wave or diverging beam scan are stored in a scan compounding memory 22, where they are combined on a spatial basis with the echo signals received from previous scans of the target volume. When the received scanlines for each transmit volume are in a common spatial distribution relative to the dimensions of its insonified pyramidal volume, convenient for beamformer programming, the scanlines from the different scans will virtually all be at different spatial angles to each other and echoes from intersection points are combined on a spatial basis. Since the time-of-flight of each echo determines its spatial position in the volume, echoes with the same x,y,z coordinates in the target volume are added together and stored in corresponding x,y,z storage locations of the scan compounding memory 22. As the echoes from each different scan volume are received, they are added to the echo data previously received from the same x,y,z locations of the target volume and stored in the memory. In this way, the echoes received from all eighty-one (or seventeen, or forty-one) volume scans of the previous examples are coherently compounded in the memory 22.

The coherent echo signals undergo signal processing by a signal processor 26, which includes filtering by a digital filter and noise or speckle reduction as by frequency compounding. The filtered echo signals also undergo quadrature bandpass filtering in the signal processor 26. This operation performs three functions: band limiting the RF echo signal data, producing in-phase and quadrature pairs (I and Q) of echo signal data, and decimating the digital sample rate. The signal processor can also shift the frequency band to a lower or baseband frequency range. The digital filter of the signal processor 26 can be a filter of the type disclosed in U.S. Pat. No. 5,833,613 (Averkiou et al.), for example.

The compounded and processed coherent echo signals are coupled to a B mode processor 30 which produces signals for a B mode image of structure in the subject such as a tissue image. The B mode processor performs amplitude (envelope) detection of quadrature demodulated I and Q signal components by calculating the echo signal amplitude in the form of (I²+Q²)^{1/2}. The quadrature echo signal components are also coupled to a Doppler processor 34. The Doppler processor 34 stores ensembles of echo signals from discrete points in an image field which are then used to estimate the Doppler shift at points in the image with a fast Fourier transform (FFT) processor. The rate at which the ensembles are acquired determines the velocity range of motion that the system can accurately measure and depict in an image. The Doppler shift is proportional to motion at points in the image field, e.g., blood flow and tissue motion. For a color Doppler image, the estimated Doppler flow values at each point in a blood vessel are wall filtered and converted to color values using a look-up table. The wall filter has an adjustable cutoff frequency above or below which motion will be rejected such as the low frequency motion of the wall of a blood vessel when imaging flowing blood. The B mode image signals and the Doppler flow values are coupled to a multiplanar reformatter 32 which extracts image signals of a desired plane of a 3D image dataset when a planar image of a scanned volume is desired. Extraction is done on the basis of the x,y,z coordinates of the 3D dataset of the tissue and flow signals, and the extracted signals are then formatted for display in a desired display format, e.g., a rectilinear display format or a sector display format. Either a B mode image or a Doppler image may be displayed alone, or the two shown together in anatomical registration in which the color Doppler overlay shows the blood flow in tissue and vessels in blood vessels of the B mode tissue image. Another display possibility is to display side-by-side images of the same anatomy which have been processed differently. This display format is useful when comparing images.

The image data is coupled to an image memory 36, where the image data is stored in memory locations addressable in accordance with the spatial locations from which the image values were acquired. Image data from 3D scanning can be accessed by a volume renderer 42, which converts the echo signals of a 3D dataset into a projected 3D image as viewed from a given reference point as described in U.S. Pat. No. 6,530,885 (Entrekin et al.) The 3D images produced by the volume renderer 42 and 2D images produced by the multiplanar reformatter 32 from a plane of a scanned volume are coupled to a display processor 48 for further enhancement, buffering and temporary storage for display on an image display 40.

A second implementation of an ultrasound imaging system of the present invention is illustrated in block diagram form in FIG. 8. Components with the same reference numerals function in the FIG. 8 implementation in the same way as in FIG. 7. The beamformer controller 118, however, instead of controlling a main system beamformer, now controls the addressing of a receiver in the form of a microchannel memory 120 in addition to its control of the microbeamformer. The microchannel memory is a 3D data memory which receives and stores the signals produced by the patches of elements of the 2D array transducer, storing them in correspondence with their locations in the scanned target volume. After all of the echo signals have been received from the target volume from a transmission of a plane wave or diverging beam, the 3D volume of data is combined on a spatial basis with the 3D data received from previous transmit events by a synthetic focus processor 122. Adding all of the echoes received from all of the plane wave or divergent transmit events on a spatial basis effects a synthetic focusing whereby image data at points throughout the volume is fully focused. See, for example, U.S. Pat. No. 4,604,697 (Luthra et al.) for a description of synthetic focusing. Similar to the previous implementation, the combining of data by the synthetic focus processor provides a compounding of the 3D datasets from the multiple plane wave or divergent scans of the target volume.

It should be noted that an ultrasound system suitable for use in an implementation of the present invention, and in particular the component structure of the ultrasound systems of FIGS. 7 and 8, may be implemented in hardware, software or a combination thereof. The various embodiments and/or components of an ultrasound system and its controller, or components and controllers therein, also may be implemented as part of one or more computers or microprocessors. The computer or processor may include a computing device, an input device, a display unit and an interface, for example, for accessing the internet. The computer or processor may include a microprocessor. The microprocessor may be connected to a communication bus, for example, to access a PACS system or the data network for importing training images. The computer or processor may also include a memory. The memory devices such as scan compounding memory 22, the image memory 36, and the microchannel memory 120 may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine. The set of instructions of an ultrasound system including those controlling the acquisition, processing, and display of ultrasound images as described above may include various commands that instruct a computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. The operation of the scan compounding memory and the synthetic focus processor are typically performed by or under the direction of software routines. Further, the software may be in the form of a collection of separate programs or modules within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasound imaging system which produces three dimensional images of a target volume comprising:
an ultrasound probe comprising a two-dimensional array of transducer elements adapted to transmit plane waves or divergent waves to the target volume and to acquire ultrasonic echo signals returned from the target volume,
a receiver, coupled to receive the echo signals from each transmission, and adapted to process the echo signals returned from the target volume on a spatial basis;
an image data compounder, coupled to the receiver, and adapted to compound image data produced in response to each transmission on a spatial basis;
an image processor, coupled to receive the compounded image data, and adapted to produce a volume image; and
a display adapted to display the volume image,
characterized in that the two-dimensional array is further adapted to transmit a plurality of such waves at different angles to the target volume, the different angles being intermediate azimuth and elevation directions relative to the array.

2. (canceled)

3. The ultrasound imaging system of claim 1, wherein the two-dimensional array is further adapted to transmit a plurality of such waves at angles which include both azimuth and elevation dimensions.

4. The ultrasound imaging system of claim 1, wherein the receiver further comprises a beamformer adapted to process received echo signals by beamformation.

5. The ultrasound imaging system of claim 4, wherein the ultrasound probe further comprises a microbeamformer, coupled to the elements of the two-dimensional array, adapted to perform partial beamforming of echo signals received by patches of array elements.

6. The ultrasound imaging system of claim 5, wherein the beamformer is further adapted to beamform partially beamformed echo signals produced by the microbeamformer.

7. The ultrasound imaging system of claim 4, wherein the image data compounder further comprises an image data memory adapted to store echo signals on a spatial basis.

8. The ultrasound imaging system of claim 1, wherein the receiver further comprises a synthetic focus processor.

9. The ultrasound imaging system of claim 8, further comprising a memory adapted to store echo signals acquired by the two-dimensional array on a spatial basis.

10. The ultrasound imaging system of claim 8, wherein the synthetic focus processor further comprises the image data compounder, and is adapted to combine echo signals received from the target volume from multiple transmissions on a spatial basis.

11. The ultrasound imaging system of claim 1, wherein the image processor further comprises a B mode processor.

12. The ultrasound imaging system of claim 1, wherein the image processor further comprises a Doppler processor.

13. The ultrasound imaging system of claim 1, wherein the image processor further comprises a multiplanar reformatter adapted to extract image data of an image plane from a 3D dataset.

14. The ultrasound imaging system of claim 1, wherein the image processor further comprises a volume renderer adapted to produce a projected image from a 3D image dataset.

15. A method for producing three dimensional images of a target volume comprising:
using an ultrasound probe comprising a two-dimensional array of transducer elements to transmit plane waves or divergent waves to the target volume and to acquire ultrasonic echo signals returned from the target volume;
receiving the echo signals from each transmission,
processing the echo signals returned from the target volume on a spatial basis;
compounding image data produced in response to each transmission on a spatial basis;
generating a volume image from the compounded image data; and
displaying the volume image,
characterized in that the method comprises using the two-dimensional array to transmit a plurality of such waves at different angles to the target volume, the different angles being intermediate azimuth and elevation directions relative to the array.
